(19) United States
(12) Patent Application Publication
(10) Pub. No.: US 2025/0265708 A1
(43) Pub. Date: Aug. 21, 2025

(54) METHOD AND SYSTEM FOR CONTRACTILITY ASSESSMENT OF CARDIAC ORGANOIDS BASED ON PARTICLE IMAGE VELOCIMETRY, PROGRAM FOR THE SAME, AND RECORDING MEDIUM STORING PROGRAM THEREOF

(71) Applicant: Research & Business Foundation SUNGKYUNKWAN UNIVERSITY, Suwon-si (KR)

(72) Inventors: So Ah LEE, Suwon-si (KR); Ho Yeon LEE, Seoul (KR); Bo Young KIM, Jinju-si (KR)

(73) Assignee: Research & Business Foundation SUNGKYUNKWAN UNIVERSITY, Suwon-si (KR)

(21) Appl. No.: 18/934,331

(22) Filed: Nov. 1, 2024

(30) Foreign Application Priority Data
Feb. 20, 2024 (KR) 10-2024-0024252

(51) Int. Cl. G06T 7/00; G06T 7/285; G06T 7/38

(52) U.S. Cl. CPC G06T 7/0012; G06T 7/285; G06T 7/38; G06T 2207/30048

(57) ABSTRACT

A PIV-based cardiac organoid contractility assessment method according to the present invention comprises the steps of: converting a captured video of a cardiac organoid into an image sequence; setting an interrogation window size and a step size for a PIV algorithm to a first value for each of the converted image sequences; configuring information on the cardiac organoid; calculating a deformation velocity of the cardiac organoid using the first value, information on the cardiac organoid, and the PIV algorithm; providing a real-time interactive tool for postprocessing of the deformation velocity; calculating multiple parameters that assess contractility of the cardiac organoid on the basis of a profile of the postprocessed deformation velocity; and outputting visual data and the multiple parameters used for analyzing contractility of the cardiac organoid, wherein the first value is reset to a second value considering continuity and calculation time of a deformation velocity vector field calculated at the step of calculating the deformation velocity of the cardiac organoid.

### CROSS REFERENCE TO RELATED APPLICATION

The present application claims priority to Korean Patent Application No. 10-2024-0024252, filed on Feb. 20, 2024, the entire contents of which is incorporated herein for all purposes by this reference.

### BACKGROUND OF THE INVENTION

### Field of the Invention

The present invention relates to assessment of contractility of cardiac organoids, more specifically, to a method, a system, and a program based on Particle Image Velocimetry (PIV), which can reliably and multifacetedly analyze contractility of 3D cardiac organoids.

### Background of the Related Art

A cardiac organoid derived from a human induced pluripotent stem cell (hiPSC) is a type of heart muscle, which is a cell collection used for developing new drugs, modeling diseases, and researching regenerative medicines. In particular, hiPSC-derived cardiac organoids are used to predict harmful effects (cardiotoxicity) and arrhythmia induced by drugs in each patient. At this point, a method of characterizing contractility of cardiac organoids is important for accurate prediction.

Several image-based analysis methods have been developed to monitor and characterize cardiac contractility. For example, the cardiac contractility analysis methods include an edge detection method, an optical motion vector analysis, and a post-deflection tracking method.

The edge detection method is a method of identifying boundaries of cardiomyocytes and assessing how the boundaries of the cardiomyocytes change over time. This method has proven useful for some cardiac models, such as a rectangular mature single cardiomyocyte (Harding et al., 1988; Steadman et al., 1988; Vescovo et al., 1989).

However, since the edge detection method measures only the changes in the edges, there is a problem in that contractility of hiPSC-derived immature cardiomyocytes or cardiac organoids of an irregular shape may not be accurately predicted.

Recently, optical motion vector analysis is widely used as an alternative. This is a method of calculating a displacement field between consecutive images to predict movement of cells. Through the optical motion vector analysis, heart-related parameters, such as heart beat frequency, maximum contraction/relaxation velocity, beating duration, and the like, can be quantified.

However, in order to apply the optical motion vector analysis, an object should be accurately defined or distinguished from the background. However, Individual cells within a 3D cardiac organoid are difficult to be accurately defined as objects because the cells overlap. In addition, there is a problem in that accuracy of the optical motion vector analysis is lowered when the size of the background or object or intensity of light changes abruptly.

Several tools have been developed to assess cardiac contractility using the optical motion vector analysis of an open source (Huebsch et al., 2015; Sala et al, 2018; Grune et al, 2019; Scalzo et al, 2021). Existing tools have following limitations. First, existing tools are validated using only 2D heart models. Second, although postprocessing tasks such as noise removal or the like are very important in analyzing a 3D heart model, as existing tools are completely automated, postprocessing tasks are not possible, or interactive tools for postprocessing data are quite limited. Third, existing tools require expensive devices such as high-frame rate cameras. Fourth, existing tools do not provide multifaceted parameters and images.

### SUMMARY OF THE INVENTION

Therefore, the present invention has been made in view of the above problems, and it is an object of the present invention to provide a contractility assessment method, system, and program that enables multifaceted contractility analysis of 3D cardiac organoid models. In addition, another object of the present invention is to provide a method, a system, and a program that can precisely analyze contractility of cardiac organoids without using a high-performance camera supporting a high frame rate.

To accomplish the above objects, according to one aspect of the present invention, there is provided a PIV-based cardiac organoid contractility assessment method and a program thereof, the method comprising the steps of: converting a captured video of a cardiac organoid into an image sequence; setting an interrogation window size and a step size for a PIV algorithm to a first value for each of the converted image sequences; configuring information on the cardiac organoid; calculating a deformation velocity of the cardiac organoid using the first value, information on the cardiac organoid, and the PIV algorithm; providing a real-time interactive tool for postprocessing of the deformation velocity; calculating multiple parameters that assess contractility of the cardiac organoid on the basis of a profile of the postprocessed deformation velocity; and outputting visual data and the multiple parameters used for analyzing contractility of the cardiac organoid, wherein the first value is reset to a second value considering continuity and calculation time of a deformation velocity vector field calculated at the step of calculating the deformation velocity of the cardiac organoid.

Here, as the second value, the PIV interrogation window size may be set to 32 pixels, and the step size may be set to 8 pixels.

In addition, an FFT algorithm may be used to perform cross-correlation with interrogation windows in the PIV algorithm.

In addition, the postprocessing may include at least any one or more among setting a noise threshold, removing noise, smoothing, deleting noise peak points, and selecting or deselecting start, peak, and end points of contraction and relaxation.

In addition, the multiple parameters may include at least any one or more among a maximum contraction force and a maximum relaxation force.

In addition, the multiple parameters may further include at least any one or more among decay times of 90%, 50%, and 10% levels.

In addition, the visual data may include a contraction-relaxation velocity profile linked to an input image in real time, a beating image overlaid with the vector field of the deformation velocity, and a beating image of a heatmap form.

In addition, the image of the cardiac organoid may be captured using a standard imaging device having a frame rate of 10 to 60 frames per second.

To accomplish the above object, according to another aspect of the present invention, there is provided a PIV-based cardiac organoid contractility assessment system comprising: a cardiac organoid image conversion unit for converting a captured video of a cardiac organoid into an image sequence; a PIV setting unit for setting an interrogation window size and a step size for a PIV algorithm to a first value for each of the converted image sequences; a cardiac organoid configuring unit for configuring information on the cardiac organoid; a deformation velocity calculation unit for calculating a deformation velocity of the cardiac organoid using the first value, information on the cardiac organoid, and the PIV algorithm; a postprocessing unit for providing a real-time interactive tool for postprocessing of the deformation velocity; a multiple parameter calculation unit for calculating multiple parameters that assess contractility of the cardiac organoid on the basis of a profile of the postprocessed deformation velocity; an output unit for outputting visual data and the multiple parameters used for analyzing contractility of the cardiac organoid; and a control unit configured of software modules, a memory, and a processor needed for operation of a computer system, wherein the first value is reset to a second value considering continuity and calculation time of a deformation velocity vector field calculated by the deformation velocity calculation unit for calculating the deformation velocity of the cardiac organoid.

Here, as the second value, the PIV interrogation window size may be set to 32 pixels, and the step size may be set to 8 pixels.

In addition, an FFT algorithm may be used to perform cross-correlation with interrogation windows in the PIV algorithm.

In addition, the postprocessing may include at least any one or more among setting a noise threshold, removing noise, smoothing, deleting noise peak points, and selecting or deselecting start, peak, and end points of contraction and relaxation.

In addition, the multiple parameters may include at least any one or more among a maximum contraction force and a maximum relaxation force.

In addition, the multiple parameters further may include at least any one or more among decay times of 90%, 50%, and 10% levels.

In addition, the visual data may include a contraction-relaxation velocity profile linked to an input image in real time, a beating image overlaid with the vector field of the deformation velocity, and a beating image of a heatmap form.

In addition, the image of the cardiac organoid may be captured using a standard imaging device having a frame rate of 10 to 60 frames per second.

### BRIEF DESCRIPTION OF THE DRAWINGS

FIG. 1 is a flowchart illustrating a method of assessing contractility of a cardiac organoid based on particle image velocimetry (PIV) according to an embodiment of the present invention.

FIG. 2A is a view for explaining the step of setting an appropriate PIV interrogation window size and step size (S120) in detail according to an embodiment of the present invention.

FIG. 2B is a view for showing the contraction-relaxation velocity of a cardiac organoid based on PIV interrogation window size 32 px and step size 8 px according to an embodiment of the present invention.

FIG. 2C is a view for showing the visualization of frame data in a cardiac organoid based on PIV interrogation window size 32 px and step size 8 px according to an embodiment of the present invention.

FIG. 3 is a view for explaining the step of calculating the deformation velocity of a cardiac organoid using a PIV algorithm (S140) in detail according to an embodiment of the present invention.

FIG. 4 is a view showing multiple parameters of an organoid according to an embodiment of the present invention.

FIG. 5 is a view showing an embodiment of the present invention for outputting visual data and multiple parameters.

FIG. 6 is a block diagram showing a system 200 for assessing contractility of cardiac organoids based on PIV according to an embodiment of the present invention.

FIG. 7A is a reference view showing results of an embodiment of the present invention for comparison with open-source contractility assessment software.

FIG. 7B is a reference view showing results of an embodiment of the present invention for comparison with open-source contractility assessment software.

FIG. 7C is a reference view showing results of an embodiment of the present invention for comparison with open-source contractility assessment software.

FIG. 7D is a reference view showing results of an embodiment of the present invention for comparison with open-source contractility assessment software.

FIG. 8A is a reference view showing statistical results of parameter ‘beating rate’ calculated for quantitative comparison and analysis of an embodiment of the present invention and comparative examples.

FIG. 8B is a reference view showing statistical results of parameter ‘contraction time-to-peak’ calculated for quantitative comparison and analysis of an embodiment of the present invention and comparative examples.

FIG. 8C is a reference view showing statistical results of parameter ‘contraction duration’ calculated for quantitative comparison and analysis of an embodiment of the present invention and comparative examples.

FIG. 8D is a reference view showing statistical results of parameter ‘time between contraction-relaxation peaks’ calculated for quantitative comparison and analysis of an embodiment of the present invention and comparative examples.

FIG. 8E is a reference view showing statistical results of parameter ‘peak contraction velocity’ calculated for quantitative comparison and analysis of an embodiment of the present invention and comparative examples.

FIG. 8F is a reference view showing statistical results of parameter ‘peak contraction velocity’ calculated for quantitative comparison and analysis of an embodiment of the present invention and comparative examples.

FIG. 8G is a reference view showing statistical results of parameter ‘contraction deformation distance’ calculated for quantitative comparison and analysis of an embodiment of the present invention and comparative examples.

FIG. 8H is a reference view showing statistical results of parameter ‘maximum contraction force’ calculated for quantitative comparison and analysis of an embodiment of the present invention and comparative examples.

FIG. 8I is a reference view showing statistical results of parameter ‘time to decay’ calculated for quantitative comparison and analysis of an embodiment of the present invention and comparative examples.

FIG. 9A is a reference view showing comparative analysis of contraction-relaxation velocity profiles at different frame rates.

FIG. 9B is a reference view showing contraction-relaxation velocity profile analyzed using a camera with 150 FPS.

FIG. 9C is a reference view showing contraction-relaxation velocity profile analyzed using a camera with 10 FPS.

FIG. 10A is a reference view showing contraction-relaxation velocity profile before drug treatment according to an embodiment of the present invention.

FIG. 10B is a reference view showing image of cardia organoid before drug treatment according to an embodiment of the present invention.

FIG. 10C is a reference view showing contraction-relaxation velocity profile 15 minutes after isoprenaline treatment according to an embodiment of the present invention.

FIG. 10D is a reference view showing image of cardia organoid 15 minutes after isoprenaline treatment according to an embodiment of the present invention.

FIG. 10E is a reference view showing contraction-relaxation velocity profile 1 day after drug withdrawal according to an embodiment of the present invention.

FIG. 10F is a reference view showing image of cardia organoid 1 day after drug withdrawal according to an embodiment of the present invention.

### DETAILED DESCRIPTION OF THE PREFERRED EMBODIMENT

Hereinafter, the present invention will be described with reference to the accompanying drawings. However, the present invention may be implemented in several different forms, and thus is not limited to the embodiments described herein. In addition, in order to clearly explain the present invention in the drawings, parts unrelated to the description are omitted, and similar reference numerals are attached to similar parts throughout the specification.

Throughout the specification, when a part is “linked (connected, contacted, coupled)” to another part, it includes the cases of being “indirectly connected” with intervention of another member therebetween, as well as the cases of being “directly connected”. In addition, when a part “includes” a certain component, this means that other components may be further provided, rather than excluding other components, unless clearly stated otherwise.

The terms used in this specification are used only to describe specific embodiments, and are not intended to limit the present invention. Singular expressions include plural expressions unless the context clearly dictates otherwise. It should be understood that in this specification, terms such as “comprise” or “have” are intended to specify existence of a feature, number, step, operation, component, part, or a combination thereof described in the specification, not to preclude the possibility of existence or addition of one or more other features, numbers, steps, operations, components, parts, or combinations thereof.

In this specification, a “module” includes a unit configured of hardware, software, or firmware, and may be used interchangeably with the terms, such as logic, logic block, part, circuit, or the like. A module may be a part configured as one body, a minimum unit that performs one or more functions, or a part thereof. For example, the module may be configured as an application-specific integrated circuit (ASIC).

Hereinafter, embodiments of the present invention will be described in detail with reference to the accompanying drawings.

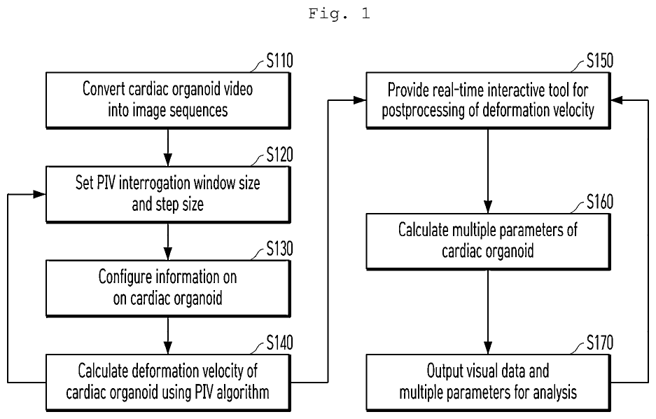

FIG. 1 is a flowchart illustrating a method of assessing contractility of a cardiac organoid based on particle image velocimetry (PIV) according to an embodiment of the present invention.

At step S110, a captured cardiac organoid contraction video I is converted into an image sequence.

Here, the contraction video I of the cardiac organoid may be captured by a camera or a microscope equipped with a camera function.

According to an embodiment of the present invention, the camera or microscope may employ even a standard imaging device generally having a frame rate of 10 to 60 frames per second.

At step S120, an interrogation window size and a step size for the PIV algorithm are set.

Specifically, the interrogation window size and the step size for the PIV algorithm may be set to a first value for each of the converted image sequences, and it is possible to change the setting to a second value through feedback of deformation velocity calculation.

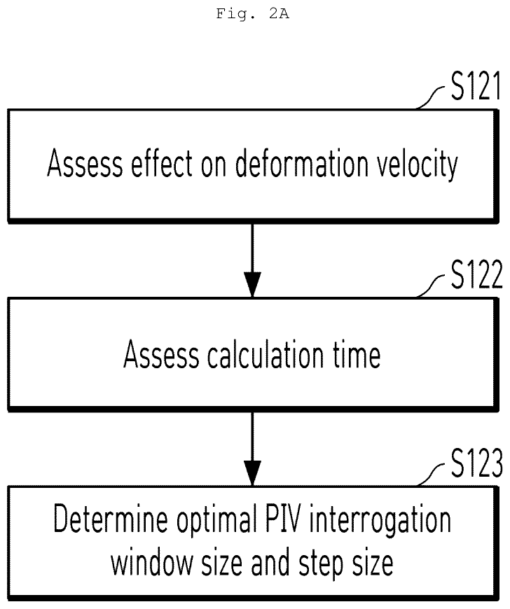

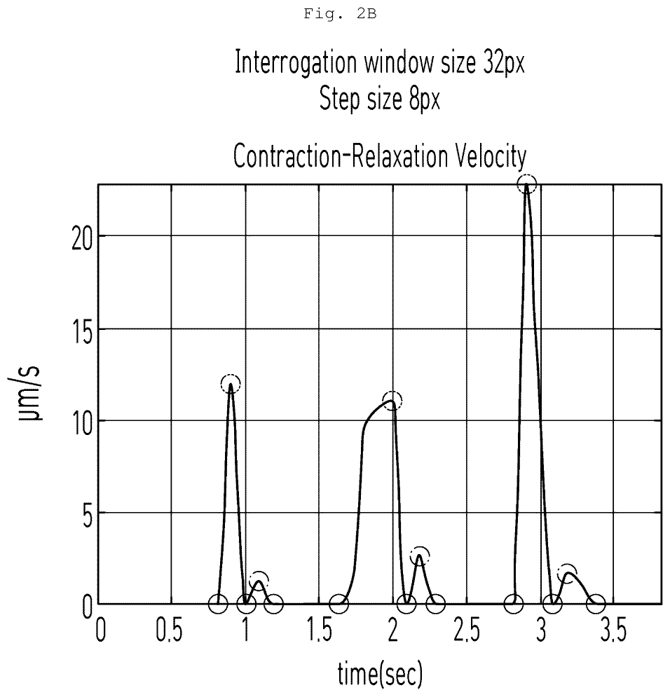

Here, setting an appropriate interrogation window size and step size may be a factor for enhancing reliability and predictability of the PIV-based contractility assessment. This will be described below in detail with reference to FIG. 2.

At step S130, information on the cardiac organoid is configured.

Information on the organoid may include stiffness and diameter. Here, stiffness of the cardiac organoid may be measured using separate equipment such as an atomic force microscope.

At step S140, the deformation velocity of the cardiac organoid is calculated using the setting values the of interrogation window size and the step size, information on the cardiac organoid, and the PIV algorithm.

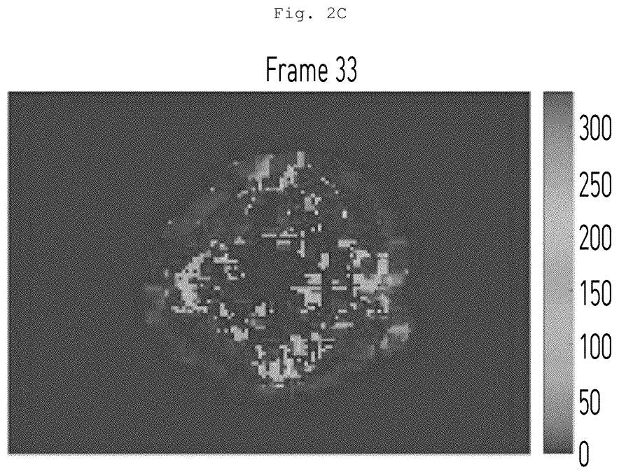

Specifically, an average deformation velocity of the cardiac organoid may be calculated using the PIV algorithm on the basis of the cardiac organoid image sequence, the set PIV interrogation window size and step size, and the stiffness and diameter of the organoid. This will be described below in detail with reference to FIG. 3.

At step S150, a real-time interactive tool for postprocessing of the deformation velocity is provided.

The postprocessing may include setting a noise threshold, removing noise, smoothing, deleting noise peak points, and selecting or deselecting start, peak, and end points of contraction and relaxation.

Although postprocessing performed automatically may be useful when the work is simple and the amount of data to be processed is large, a manual postprocessing work performed by an expert through a real-time interactive tool is required in analyzing 3D cardiac organoids, in which it is difficult to automatically distinguish noise peaks from actual beating peaks.

At step S160, multiple parameters for assessing contractility of the cardiac organoid are calculated on the basis of the profile of the postprocessed deformation velocity.

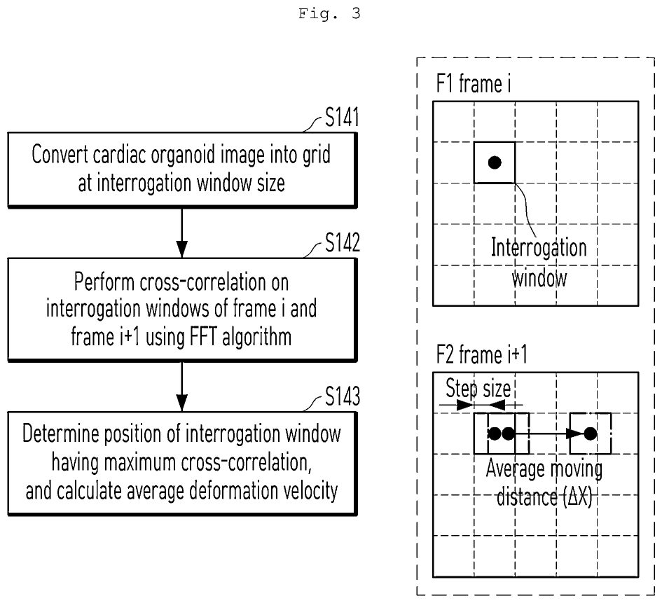

Here, the multiple parameters will be described below in detail with reference to FIG. 4.

At step S170, visual data and multiple parameters used for analyzing contractility of the cardiac organoid are output.

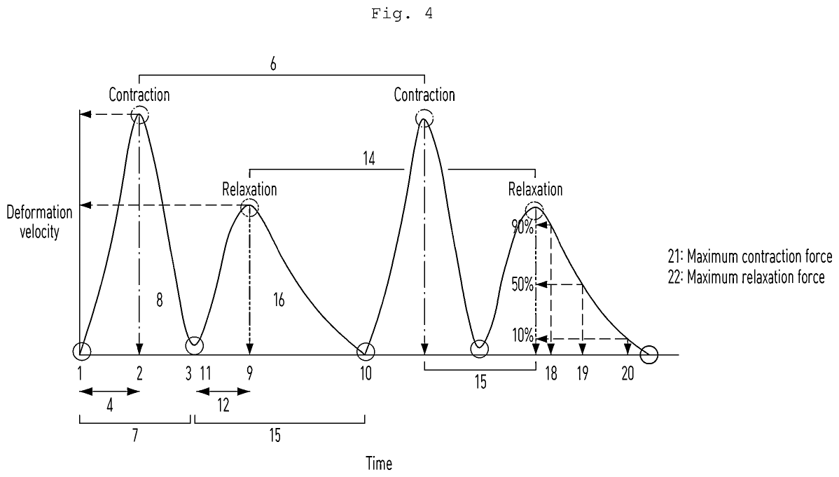

Here, the output visual data and multiple parameters will be described below in detail with reference to FIG. 5.

FIG. 2A is a view for explaining the step of setting an appropriate PIV interrogation window size and step size (S120) in detail according to an embodiment of the present invention.

At step S121, the user sets various interrogation window sizes and step sizes, and assesses the effect on the deformation velocity calculated therefrom at step S140.

Particularly, the effect of the interrogation window size and step size on the deformation velocity may be assessed based on when the cardiac organoid contracts the most.

Here, as the interrogation window size or the step size increases, the deformation velocity appears discontinuous in the vector field due to the stiff difference in the velocity between adjacent interrogation windows, and on the contrary, as the interrogation window size or the step size decreases, the deformation velocity tends to be shown more continuously and densely in the vector field.

At step S122, the user assesses the time required to calculate the deformation velocity.

Here, the calculation time tends to increase as the interrogation window size or the step size decreases, and conversely, the calculation time tends to increase as the interrogation window size or the step size increases.

At step S123, the user determines an optimal PIV interrogation window size and step size by comprehensively considering the effect on the deformation velocity and calculation time. The optimal interrogation window size and step size may be reset in a trial-and-error manner while considering continuity of the deformation velocity vector field and the calculation time.

FIG. 2B is a view for showing the contraction-relaxation velocity of a cardiac organoid based on PIV interrogation window size 32 px and step size 8 px according to an embodiment of the present invention.

FIG. 2C is a view for showing the visualization of frame data in a cardiac organoid based on PIV interrogation window size 32 px and step size 8 px according to an embodiment of the present invention.

According to an embodiment of the present invention, as shown in FIG. 2B and FIG. 2C, the optimal interrogation window size may be set to 32 pixels and the optimal step size may be set to 8 pixels for analysis of cardiac organoid contractility.

FIG. 3 is a view for explaining the step of calculating the deformation velocity of a cardiac organoid using a PIV algorithm (S140) in detail according to an embodiment of the present invention.

At step S141, each cardiac organoid image sequence is converted into a grid of the interrogation window size set by the user.

At step S142, the interrogation window of frame i F1 is cross-correlated with the interrogation windows of a series of i+1 frames F2 moved as much as the step size set by the user. The cross-correlation process may be performed by the piv_FFTmulit function of PIVlab, which is an open-source toolbox of MATLAB.

Specifically, the cross-correlation is calculated by the Fast Fourier Transform (FFT) algorithm. The FFT algorithm may more accurately calculate the cross-correlation of the interrogation window by transforming the image from a spatial domain to a wavenumber domain.

At step S143, the position of the interrogation window having the maximum cross-correlation is determined through the cross-correlation, and a deformation velocity is calculated.

Specifically, the deformation velocity may be determined on average by estimating the moving distance and direction through the time difference between frame i and frame i+1 and the average position difference (displacement) of the interrogation window having the maximum cross correlation.

Meanwhile, since existing optical motion vector analysis methods are used to estimate movement of objects or features in an image sequence and assume that the intensity pattern of the image changes smoothly over time, accuracy of analysis may be lowered in a situation including abrupt changes in the shape of the object and intensity of pixels constituting the object, and technical difficulties exist in accurately tracking the movement of objects in an image when the objects overlap each other and are difficult to distinguish from the background. Therefore, in the cardiac organoids of 3D model, the existing optical motion vector analysis methods have difficulties in analyzing contractility due to deformation in the shape of cells and overlap of cells during contraction.

On the other hand, the embodiment of the present invention may calculate movement of cardiac organoids on the basis of similarity of patterns in the interrogation window. When an ordinary PIV algorithm is used in the field of fluid mechanics, small fluorescent particles are included in the fluid to introduce a ‘pattern’. However, in the embodiment of the present invention, the displacement may be tracked without fluorescent particles by allowing unique characteristics of cells or distribution patterns of cells in the cardiac organoids to function as identifiable unique patterns.

In addition, in the embodiment of the present invention, as calculation may be performed more accurately than in the spatial domain by converting the image pattern in the spatial domain into the image pattern in the frequency domain using the FTT in a 3D cardiac organoid model, dynamic processes such as contraction and relaxation can be analyzed more precisely than using existing tools.

FIG. 4 is a view showing multiple parameters of an organoid according to an embodiment of the present invention.

In FIG. 4, parameter 1 is the contraction start point, parameter 2 is the contraction peak point, parameter 3 is the contraction end point, parameter 4 is the contraction time to peak, parameter 5 is the contraction velocity, parameter 6 is the contraction heart beating rate, parameter 7 is the contraction duration, parameter 8 is the contraction deformation distance, parameter 9 is the relaxation peak point, parameter 10 is the relaxation end point, parameter 11 is the relaxation start point, parameter 12 is the relaxation time to peak, parameter 13 is the relaxation velocity, parameter 14 is the relaxation heart beating rate, parameter 15 is the relaxation duration, parameter 16 is the relaxation deformation distance, parameter 17 is the contraction-relaxation time, parameters 18, 19, and 20 are decay times (time-to-decay) of 90%, 50%, and 10% levels, respectively, parameter 21 is the maximum contraction force, and parameter 22 is the maximum relaxation force.

Here, the contraction force and relaxation force generated in the cardiac organoid may be calculated from Equations 1, 2, and 3 shown below by applying the theory of elasticity.

$$K = \frac{- Δ P}{Δ V / {V}_{0}} \qquad [ equation 1 ]$$

Here, K denotes the bulk modulus of organoid, ΔV denotes the change in the volume of organoid, and V₀ denotes the initial volume of organoid.

$$σ = K \times \frac{Δ V}{{V}_{0}} \approx 3 \times E \times \frac{( \frac{4 π}{3} Δ {r}^{3} )}{( \frac{4 π}{3} {r}_{0}^{3} )} \qquad [ equation 2 ]$$

Here, σ denotes the contraction stress, E denotes the elastic modulus of organoid, r₀ denotes the initial radius of organoid, and Δr denotes the change in the radius of organoid, which can be calculated from the average deformation distance calculated based on PIV.

The elastic modulus E may be determined by quantitatively analyzing the Force-Distance (FD) curve.

$$F = σ \times A \qquad [ equation 3 ]$$

Here, F denotes the contraction force of organoid, and A denotes the surface area of organoid.

FIG. 5 is a view showing an embodiment of the present invention for outputting visual data and multiple parameters.

Specifically, using an image that captures beating of the cardiac organoid as input data, a profile (O1) indicating the contraction-relaxation velocity linked to the input image in real time may be generated and output, a vector field of deformation velocity calculated based on PIV may be overlaid on the cardiac organoid beating image and output as a video (left side of O2), an organoid beating image may be output in the form of a heatmap that visualizes the intensity of the deformation velocity (right side of O2), and 22 multiple parameters calculated as described above may be output.

As a result, the embodiment of the present invention may provide a multifaceted profile for the contraction-relaxation velocity and provide improved visual data for assessing contractility of cardiac organoids by outputting 22 parameters for assessing contractility of the cardiac organoid.

Through the provided data, the user may return to step S150 and perform postprocessing again, such as resetting the noise threshold, removing noise, deleting noise peaks, and adjusting the start, peak, and end points of contraction and relaxation by reselecting or deselecting with reference to the overlaid cardiac organoid beating image and the heatmap video, and write comments.

FIG. 6 is a block diagram showing a system 200 for assessing contractility of cardiac organoids based on PIV according to an embodiment of the present invention.

A cardiac organoid image conversion unit 210 receives a cardiac organoid contraction video I and converts it into an image sequence.

Here, the contraction video I of the cardiac organoid may be captured by a camera or a microscope equipped with a camera function.

According to an embodiment of the present invention, the camera or microscope may also be employed as a standard imaging device generally having a frame rate of 10 to 60 frames per second.

The PIV setting unit 220 sets an interrogation window size and a step size for the PIV algorithm.

Specifically, the interrogation window size and the step size for the PIV algorithm are set to a first value for each of the converted image sequences.

Here, setting an appropriate interrogation window size and step size may be a factor for enhancing reliability and predictability of the PIV-based contractility assessment.

An optimal PIV interrogation window size and step size are determined by comprehensively considering the effect on the deformation velocity and calculation time. The optimal interrogation window size and step size may be reset in a trial-and-error manner while considering continuity of the deformation velocity vector field and the calculation time.

According to an embodiment of the present invention, as shown in E1 and E2 of FIG. 2, it is preferable that the optimal interrogation window size is set to 32 pixels and the optimal step size is set to 8 pixels for analysis of cardiac organoid contractility.

A cardiac organoid configuring unit 230 configures information on the cardiac organoid.

Information on the organoid may include stiffness and diameter. Here, stiffness of the cardiac organoid may be measured using separate equipment such as an atomic force microscope.

A deformation velocity calculation unit 240 calculates the deformation velocity of the cardiac organoid using the setting values of the interrogation window size and the step size, information on the cardiac organoid, and the PIV algorithm.

Specifically, an average deformation velocity of the cardiac organoid may be calculated using the PIV algorithm on the basis of the cardiac organoid image sequence, the set PIV interrogation window size and step size, and the stiffness and diameter of the organoid.

First, each cardiac organoid image sequence is gridded at the interrogation window size set by the user.

Next, the interrogation window of frame i F1 is cross-correlated with the interrogation windows of a series of i+1 frames F2 moved as much as the step size set by the user. The cross-correlation process may be performed by the piv_FFTmulit function of PIVlab, which is an open-source toolbox of MATLAB.

Specifically, the cross-correlation is calculated by the Fast Fourier Transform (FFT) algorithm. The FFT algorithm may more accurately calculate the cross-correlation of the interrogation window by transforming the image from a spatial domain to a wavenumber domain.

Next, the position of the interrogation window having the maximum cross-correlation is determined through the cross-correlation, and an average deformation velocity is calculated.

Specifically, the deformation velocity may be determined on average by estimating the moving distance and direction through the time difference between frame i and frame i+1 and the average position difference (displacement) of the interrogation window having the maximum cross correlation.

Meanwhile, since existing optical motion vector analysis methods are used to estimate movement of objects or features in an image sequence and assume that the intensity pattern of the image changes smoothly over time, accuracy of analysis may be lowered in a situation including abrupt changes in the shape of the object and intensity of pixels constituting the object, and technical difficulties exist in accurately tracking the movement of objects in an image when the objects overlap each other and are difficult to distinguish from the background. Therefore, in the cardiac organoids of 3D model, the existing optical motion vector analysis methods have difficulties in analyzing contractility due to deformation in the shape of cells and overlap of cells during contraction.

On the other hand, the embodiment of the present invention may calculate movement of cardiac organoids on the basis of similarity of patterns in the interrogation window. When an ordinary PIV algorithm is used in the field of fluid mechanics, small fluorescent particles are included in the fluid to introduce a ‘pattern’. However, in the embodiment of the present invention, the displacement may be tracked without fluorescent particles by allowing unique characteristics of cells or distribution patterns of cells in the cardiac organoids to function as identifiable unique patterns.

In addition, in the embodiment of the present invention, as calculation may be performed more accurately than in the spatial domain by converting the image pattern in the spatial domain into the image pattern in the frequency domain using the FTT in a 3D cardiac organoid model, dynamic processes such as contraction and relaxation can be analyzed more precisely than using existing tools.

A postprocessing unit 250 provides a real-time interactive tool for postprocessing of the deformation velocity.

The postprocessing may include setting a noise threshold, removing noise, smoothing, deleting noise peak points, and selecting or deselecting start, peak, and end points of contraction and relaxation.

Although postprocessing performed automatically may be useful when the work is simple and the amount of data to be processed is large, a manual postprocessing work performed by an expert through a real-time interactive tool is required in analyzing 3D cardiac organoids, in which it is difficult to automatically distinguish noise peaks from actual beating peaks.

A multiple parameter calculation unit 260 calculates multiple parameters for assessing contractility of the cardiac organoid on the basis of the profile of the postprocessed deformation velocity.

As shown in FIG. 4, 22 multiple parameters may be calculated. Here, the process of calculating the maximum contraction force and relaxation force is the same as described above.

The output unit 270 outputs visual data and multiple parameters used for analyzing contractility of the cardiac organoid.

Specifically, as shown in FIG. 5, using an image that captures beating of the cardiac organoid as input data, the output unit 270 may generate and output a profile (O1) indicating the contraction-relaxation velocity linked to the input image in real time, overlay a vector field of deformation velocity calculated based on PIV on the cardiac organoid beating image and output as a video (left side of O2), output an organoid beating image in the form of a heatmap that visualizes the intensity of the deformation velocity (right side of O2), and output 22 multiple parameters calculated as described above.

As a result, the embodiment of the present invention may provide a multifaceted profile for the contraction-relaxation velocity and provide improved visual data for assessing contractility of cardiac organoids by outputting 22 parameters for assessing contractility of the cardiac organoid.

Through the provided data, the postprocessing unit 250 may perform postprocessing again, such as resetting the noise threshold, removing noise, deleting noise peaks, and adjusting the start, peak, and end points of contraction and relaxation by reselecting or deselecting with reference to the overlaid cardiac organoid beating image and the heatmap video, in response to a request of the user, and the multiple parameter calculation unit 260 and the output unit 270 may recalculate the multiple parameters by reflecting the result of the postprocessing performed again, and output again the visual data and the multiple parameters.

The control unit 280 represents a memory that stores a collection of software modules, instruction sets, and application data needed for operation of a computer system, and a processor that performs tasks.

The configuration like this is only an example, and according to the environment in which the configuration is used, some configurations may be omitted, or a mobile media connection unit, a network connection unit, or the like may be added.

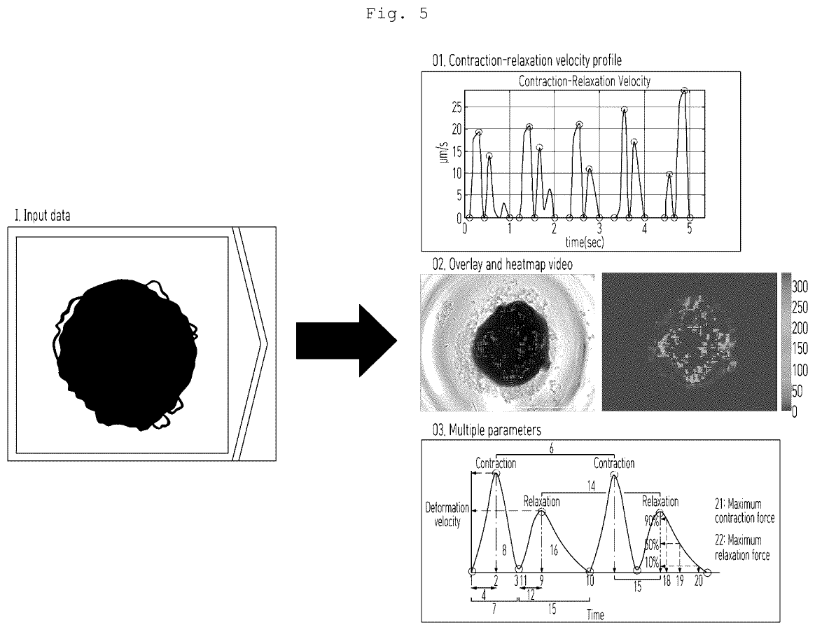

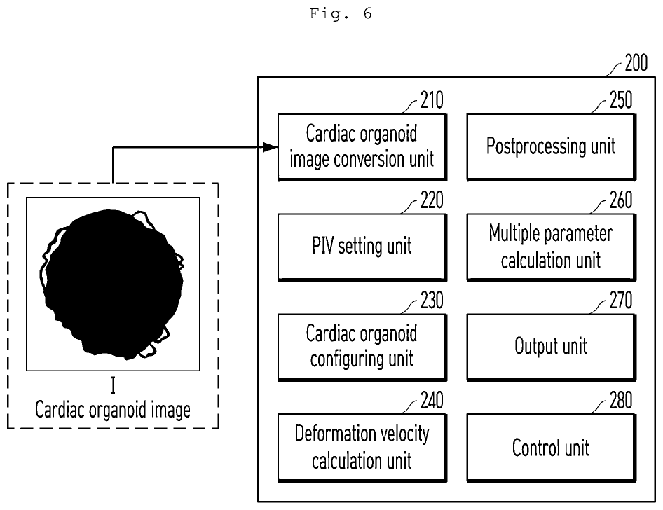

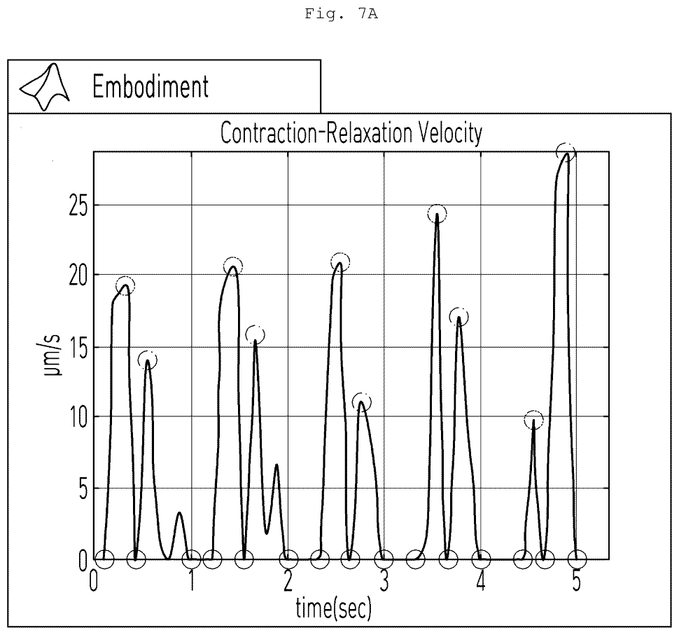

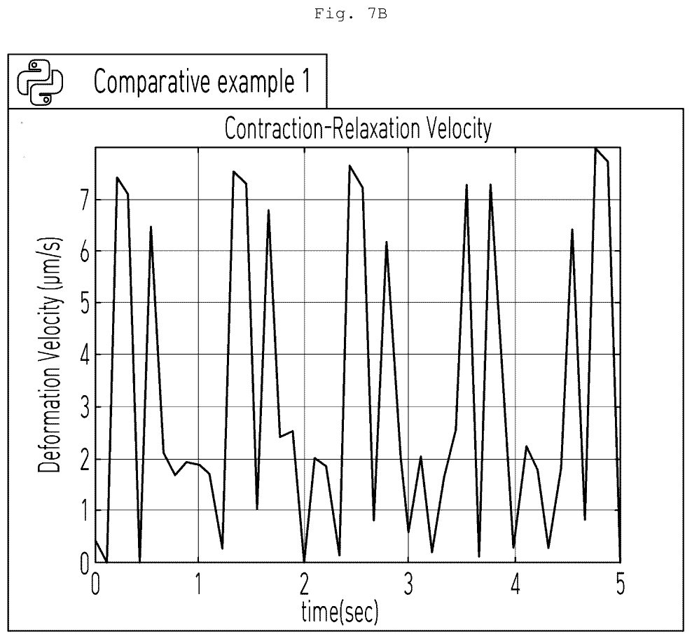

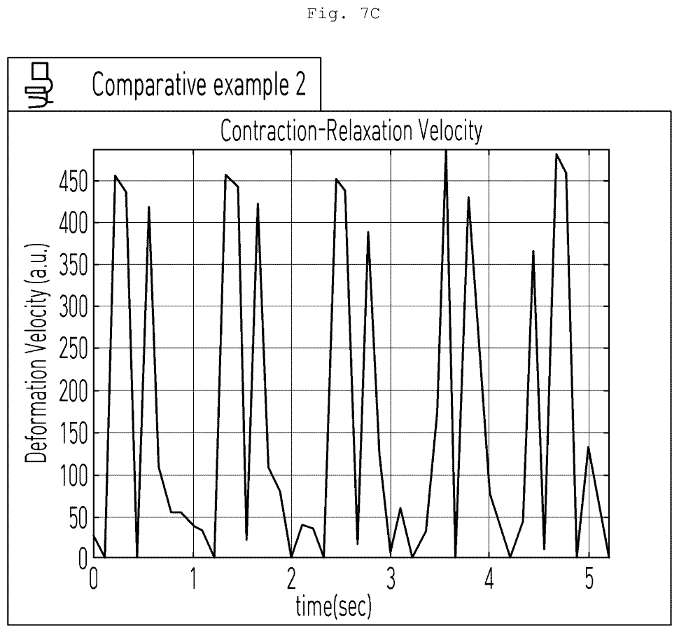

FIG. 7A, FIG. 7B, FIG. 7C and FIG. 7D are reference views showing results of comparing an embodiment of the present invention with open-source contractility assessment software.

In order to observe performance of the contractility assessment of the embodiment of the present invention, open-source contractility assessment software and contractility assessment results are compared. Four pieces of open-source contractility assessment software used for comparison of Comparative Examples 1 to 4 are ContractionWave, MUSCLEMOTION, MYOCYTER, and MotionGUI (Huebsch et al., 2015; Sala et al, 2018; Grune et al, 2019; Scalzo et al. al, 2021).

FIG. 7A shows the contraction-relaxation velocity profile of a cardiac organoid according to an embodiment of the present invention. Comparing the contraction-relaxation velocity profiles of Comparative Example 1 FIG. 7B, Comparative Example 2 FIG. 7C, and Comparative Example 3 FIG. 7D, which assess contractility on the same cardiac organoid image, it can be confirmed that patterns of similar heart beating rate appear.

For reference, in the case of Comparative Example 4, as there is no function to access raw data for acquiring the contraction-relaxation velocity profile, it may not be visually output.

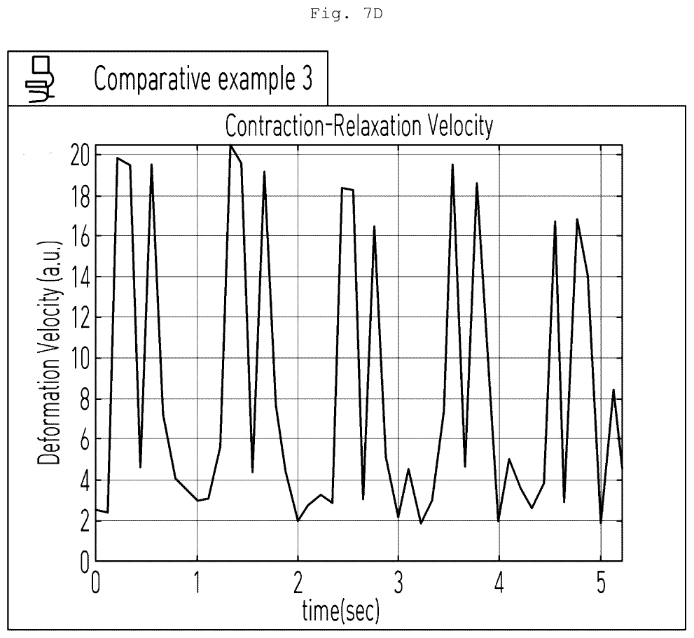

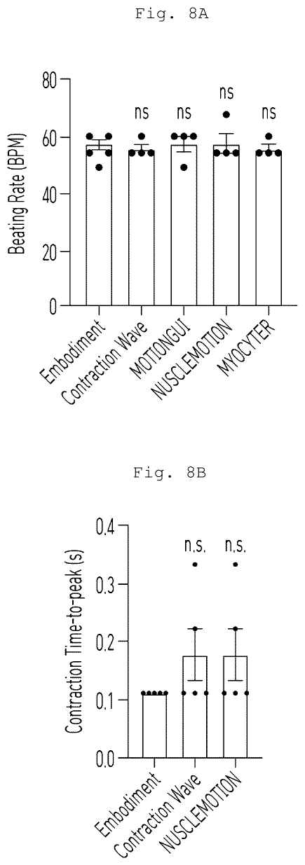

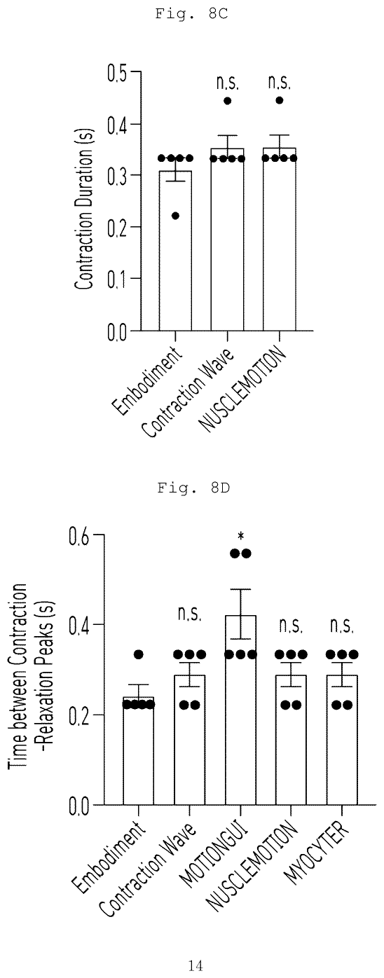

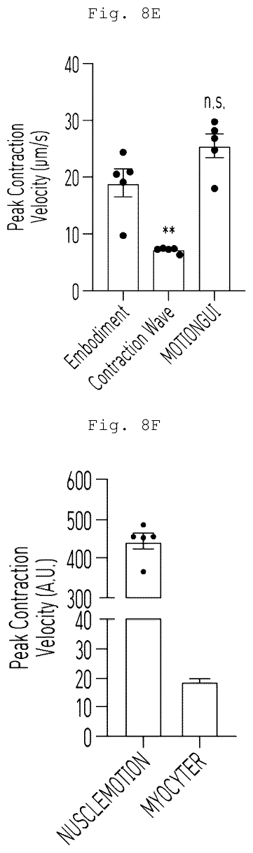

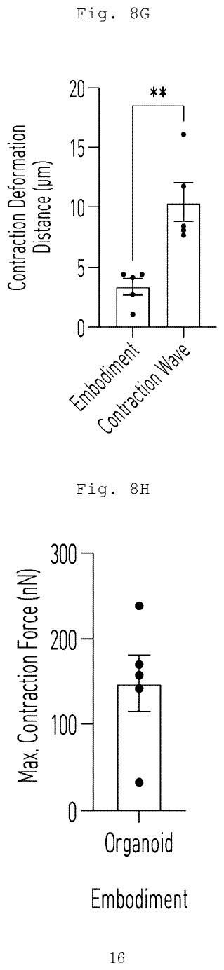

FIG. 8A through FIG. 8I are reference views showing statistical results of parameters, including beating rate, contraction time-to-peak, contraction duration, time between contraction-relaxation peaks, peak contraction velocity, contraction deformation distance, maximum contraction force, and time to decay) which were calculated for the quantitative comparison and analysis of an embodiment of the present invention and comparative examples.

Observing parameters such as heart beating rate, contraction time to peak, contraction duration, time between contraction-relaxation peakscontraction velocity in FIG. 8A through FIG. 8D, it can be confirmed that there is no difference statistically (not significant, n.s.) between the value calculated in the embodiment and the values calculated in the comparative examples. This indicates that the contraction and relaxation periods of the cardiac organoid can be successfully analyzed through the embodiment of the present invention.

In addition, some parameters, such as ‘contraction time-to-peak’ and ‘contraction duration’, may not be output in Comparative Examples 3 and 4, and parameter results shown as hatched bars are not parameters automatically calculated in the comparative examples, but parameters that the user should calculate manually.

In addition, there is an advantage in that only the embodiment of the present invention and Comparative Example 1 may output important parameter ‘contraction deformation distance’ having a positive correlation with the contraction work, and only the embodiment of the present invention may output parameters ‘time-to-decay’ and parameter ‘maximum contraction force’.

For reference, in parameter ‘contraction velocity’, the values of the embodiment of the present invention and Comparative Example 4 are similar. Although the values of comparative Examples 2 and 3 appear to be different from the value of the embodiment of the present invention, direct comparison thereof is difficult since an arbitrary unit expressed for comparison in an experimental context different from that of the embodiment is used. However, it should be noted that parameter ‘peak contraction velocity’ shown in the embodiment appears to be different from that of Comparative Example 1.

As a result, the embodiment of the present invention may output 22 quantitative parameters unlike the comparative examples, and provide a comprehensive profile and improved visual data of contractility of the cardiac organoid.

Here, the contraction deformation distance is an indirect parameter that may assess contractility, the decay time is a parameter related to Ca2+ recycling, and the contraction time to peak reflects the depolarization rate. Outputting these additional parameters may be helpful for identifying potential cellular mechanisms affected by genetic and pharmacological perturbation.

A comprehensive comparison between the embodiment of the present invention and the four comparative examples is shown below in Table 1.

TABLE 1

| | | Comparative | Comparative | Comparative | Comparative |
|---|---|---|---|---|---|
| Category | Embodiment | Example 1 | Example 2 | Example 3 | Example 4 |
| Interface | Console and GUI | GUI | ImageJ interface | ImageJ interface | GUI |
| Contractility | PIV | Optical motion vector | Optical motion vector | | Block matching |
| assessment method | | | | | using macro blocks |
| Manual noise removal | ◯ | X | X | X | X |
| Manual selection of peak | ◯ | X | X | X | X |
| Manual selection of start | ◯ | ◯ | X | X | X |
| and end points | | | | | |
| Raw data provided | ◯ | ◯ | ◯ | ◯ | X |
| Heart beating rate provided | ◯ | Δ (manual) | Δ (manual) | Δ (manual) | ◯ |
| Time-to-decay provided | ◯ | X | X | X | X |
| Contraction/relaxation | ◯ | ◯ | ◯ | X | X |
| duration provided | | | | | |
| Peak point provided | ◯ | ◯ | ◯ | X | X |
| Maximum contraction | ◯ | X | X | X | X |
| force provided | | | | | |
| Maximum speed provided | ◯ (μm/s) | ◯ (μm/s) | Δ (a.u.) | Δ (a.u.) | ◯ (μm/s) |
| (unit) | | | | | |
| Deformation distance (unit) | ◯ (μm) | ◯ (μm) | Δ (a.u.) | Δ (a.u.) | X |
| Profile output | ◯ | X | ◯ | ◯ | X |
| Overlay video output | ◯ | X | X | Δ (FPS altered) | X |
| Heatmap video output | ◯ | Δ (sequence) | X | X | X |
| Velocity vector field | ◯ | Δ (sequence) | X | X | ◯ |
| video output | | | | | |

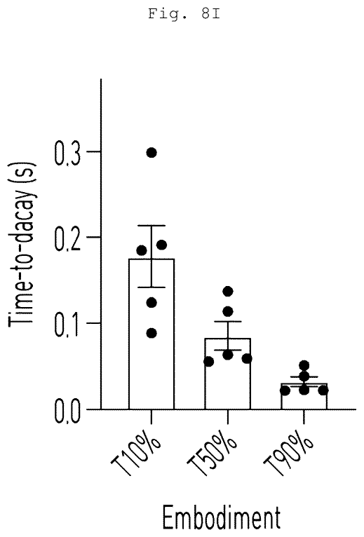

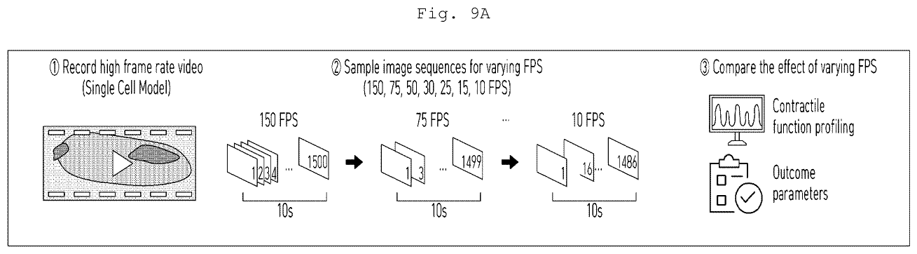

FIG. 9A is a reference view showing comparative analysis of contraction-relaxation velocity profiles at different frame rates.

Specifically, according to an embodiment of the present invention, the effect on the result of analyzing contractility of the cardiac organoid according to the change in the frame rate is observed by capturing the beating image of a single hiPSC-derived cardiac organoid using a high-performance camera (150 FPS) and generating videos of lower frame rates (75, 50, 30, 25, 15, 10 FPS) by sampling image sequences.

As a result, FIG. 9B shows a contraction-relaxation velocity profile analyzed using a camera of 150 FPS, and FIG. 9C shows a contraction-relaxation velocity profile analyzed using a camera of 10 FPS. According to an embodiment of the present invention, it can be confirmed that the contraction-relaxation velocity profile pattern is not distorted or significantly changed despite the change in the frame rates.

That is, since high-performance cameras are not provided in some environments, the present invention may allow cost-effective analysis even by using only relatively low-performance standard experiment equipment.

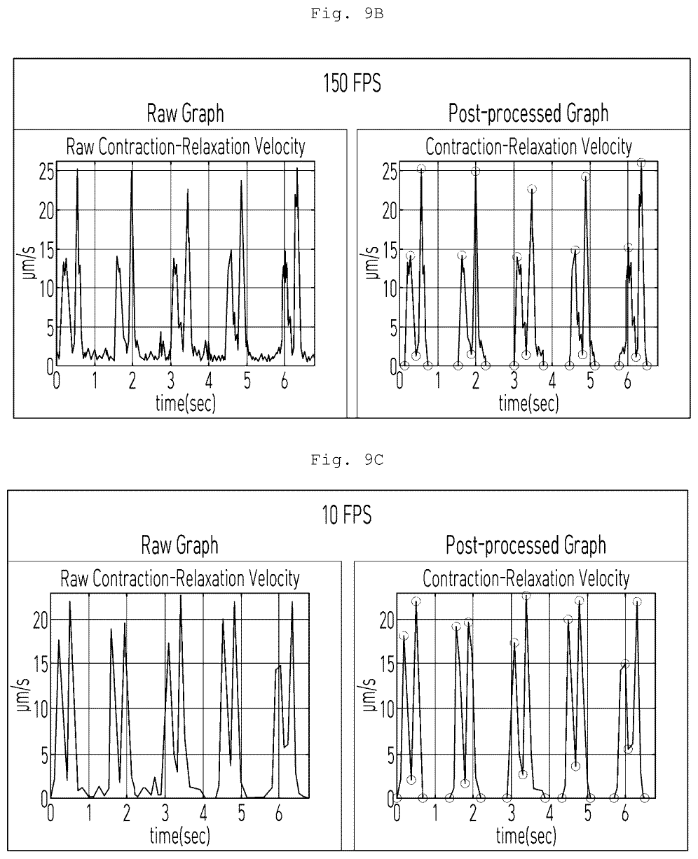

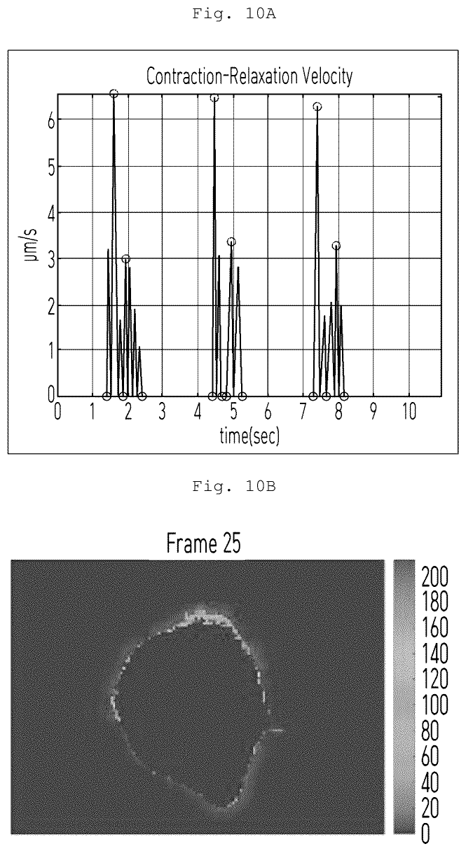

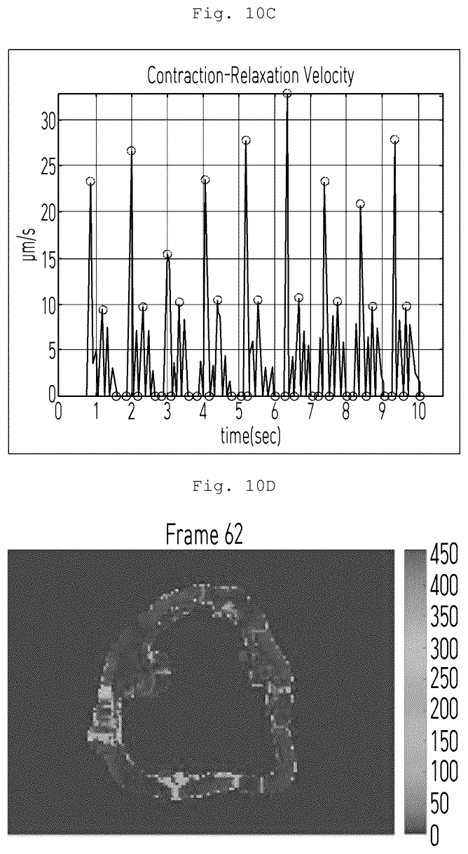

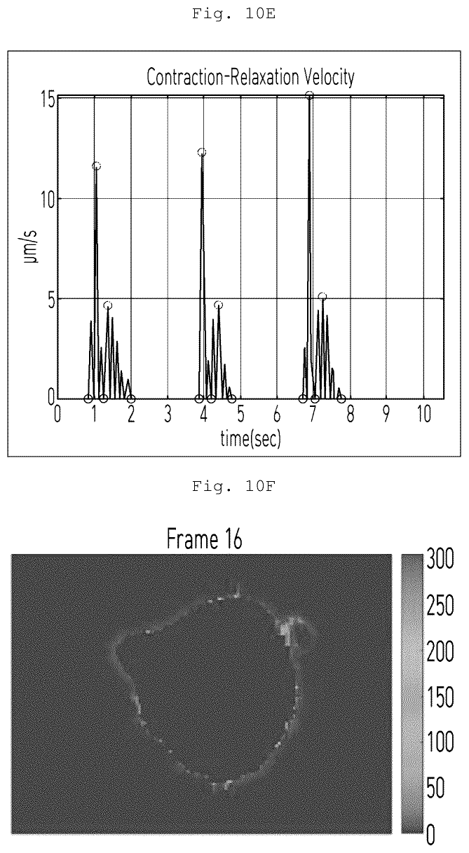

FIG. 10A through FIG. 10F is a reference view showing results of contractility analysis in a drug reaction experiment according to an embodiment of the present invention.

Specifically, isoprenaline, which is a widely used diuretic drug (inotropic agent), is used to assess contractility of a cardiac organoid in response to drugs. Here, the isoprenaline is a beta-adrenergic receptor agonist that shows positive chronotropic and diuretic effects. i) Before treating the cardiac organoid with the drug isoprenaline, a beating image of the heart organoid is captured ii) after treating for 15 minutes and iii) 1 day after drug withdrawal, and contractility is analyzed using the embodiment of the present invention.

As a result, the contraction-relaxation velocity profile and beating image of the cardiac organoids before drug treatment may be confirmed as shown in FIG. 10A and FIG. 10B, after drug treatment as shown in FIG. 10C and FIG. 10D, and 1 day after drug withdrawal as shown in FIG. 10E and FIG. 10F. Before the drug treatment, the cardiac organoid shows an average beating rate of 24.3±3.22 BPM. After isoprenaline drug treatment, the heart beating rate is increased significantly, the average contraction duration is reduced, and the contraction time to peak is also reduced. It can be confirmed that 1 day after drug withdrawal, heart beating of the organoid has returned to before the drug treatment.

Therefore, according to an embodiment of the present invention, the present invention may be a useful tool and method capable of profiling contractility of cardiac organoids in a long-term and non-destructive manner in the reaction of drug such as isoprenaline, and analyzing long-and-short term drug effects on the heart.

In conclusion, the present invention may overcome the limitations of existing optical motion analysis methods and analyze contractility of 3D cardiac organoids more precisely.

In addition, the present invention has been repeatedly demonstrated to set an interrogation window size and a step size optimized to a cardiac organoid model, and in the case of the cardiac organoid model, it is confirmed that contractility can be accurately quantified while maintaining a reasonable calculation time when an interrogation window size of 32 pixels and a step size of 8 pixels are used.

In addition, the present invention has following advantages compared to Comparative Examples 1 to 4. First, contractility of a cardiac organoid can be analyzed using a camera with a low frame rate. Second, a total of 22 parameters may be output by providing additional parameters. Third, improved analysis visual data can be provided. Finally, the present invention may provide a real-time interactive tool for performing postprocessing.

The PIV-based cardiac organoid contractility assessment method, system, and program according to an embodiment of the present invention may overcome the limitations of existing optical motion vector analysis methods and analyze contractility more precisely in a 3D cardiac organoid model by assessing contractility using a PIV algorithm.

In addition, according to an embodiment of the present invention, as an interrogation window size and a step size optimized to the PIV algorithm are set, contractility of a cardiac organoid can be accurately assessed while maintaining a short calculation time.

In addition, according to an embodiment of the present invention, as postprocessing is performed by providing a real-time interactive tool that a user may use in analyzing 3D cardiac organoids, in which noise peaks cannot be automatically distinguished from real peaks, accuracy can be improved.

In addition, according to an embodiment of the present invention, accurate analysis can be performed cost-effectively by using only standard experiment equipment rather than a camera with a high frame rate.

In addition, in an embodiment of the present invention, as 22 multiple parameters are output, multifaceted and quantitative assessment results can be provided for the contractility of cardiac organoids, and as improved images and profiles are output, visual data can be analyzed easily.

In addition, an embodiment of the present invention may be a useful method and tool capable of assessing contractility of cardiac organoids in a long-term and non-destructive manner in a drug reaction experiment and analyzing long-and-short term drug effects on the heart.

The effects of the present invention are not limited to the effects described above, and should be understood to include all effects that can be inferred from the configuration of the present invention described in the detailed description or claims of the present invention.

The method according to an embodiment of the present invention described above may be implemented in the form of program instructions that can be executed through various computer components, and recorded in a computer-readable recording medium. The computer-readable recording medium may include program instructions, data files, data structures, and the like alone or in combination. The program instructions recorded in the computer-readable recording medium may be specially designed and configured for the embodiments of the present invention, or may be known and available to those skilled in the art of computer software field. The computer-readable recording medium includes hardware configured to store and execute program instructions, such as a magnetic recording medium including a hard disk, a floppy disk, and a magnetic tape, an optical recording medium including a CD-ROM and a DVD, a magneto-optical medium including a floptical disk, ROM, RAM, flash memory, and the like. The program instructions include machine codes generated by a compiler, and high-level language codes that can be executed in a computer using an interpreter. The hardware may be configured to operate by one or more software modules to process the method according to the present invention, and vice versa.

The method according to an embodiment of the present invention may be executed in an electronic device in the form of a program instruction. The electronic device includes a portable communication device such as a smart phone, a smart pad or the like, a computer device, a portable multimedia device, a portable medical device, a camera, a wearable device, and a home appliance device.

The method according to an embodiment of the present invention may be provided to be included in a computer program product. The computer program product is a merchandise and may be traded between sellers and buyers. The computer program product may be distributed in the form of a device-readable recording medium or online through an application store. In the case of online distribution, at least some of the computer program products may be temporarily stored or temporarily generated in a storage medium such as a memory of a manufacturer server, an application store server, or a relay server.

Each of the components, for example, a module or a program, according to an embodiment of the present invention may be configured as a single sub-component or a plurality of sub-components, and some of the sub-components may be omitted, or other sub-components may be further included. Some components (modules or programs) may be integrated into a single entity to perform the same or similar functions performed by each corresponding component before the integration. Operations performed by the modules, programs, or other components according to an embodiment of the present invention may be executed sequentially, in parallel, repetitively, or heuristically, or at least some of the operations may be executed in a different order or omitted, or other operations may be added.

The description of the present invention described above is for illustrative purposes, and those skilled in the art may understand that it can be easily deformed into other specific forms without changing the technical spirit or essential features of the present invention. Therefore, it should be understood that the embodiments described above are illustrative in all respects and not restrictive. For example, each component described as a single type may be implemented in a distributed form, and components described as distributed may also be implemented in a combined form likewise.

The scope of the present invention is indicated by the following claims, and all changes or modifications derived from the meaning and scope of the claims and their equivalents should be construed as being included in the scope of the present invention.

### DESCRIPTION OF SYMBOLS

E1: Contraction-relaxation velocity profile with interrogation window size of 32 pixels and step size of 8 pixels
E2: Deformation velocity vector field when interrogation window size is 32 pixels and step size is 8 pixels
F1: Interrogation window size of frame i
F2: Interrogation window size and step size of i+1 frame
1: Contraction start point
2: Contraction peak point
3: Contraction end point
4: Contraction time to peak
5: Contraction velocity
6: Contraction heart beating rate
7: Contraction duration
8: Contraction deformation distance
9: Relaxation peak point
10: Relaxation end point
11: Relaxation start point
12: Relaxation time to peak
13: Relaxation velocity
14: Relaxation heart beating rate
15: Relaxation duration
16: Relaxation deformation distance
17: Contraction-relaxation time
18: Decay time of 90% level
19: Decay time of 50% level
20: Decay time of 10% level
21: Maximum contraction force
22: Maximum relaxation force
I: Input data
O1: Contraction-relaxation velocity profile
O2: Overlay and heatmap video
O3: Multiple parameters
200: PIV-based cardiac organoid contractility assessment system
210: Cardiac organoid image conversion unit
220: PIV setting unit
230: Cardiac organoid configuring unit
240: Deformation velocity calculation unit
250: Postprocessing unit
260: Multiple parameter calculation unit
270: Output unit
280: Control unit

What is claimed is:

1. A PIV-based cardiac organoid contractility assessment method comprising the steps of:
converting a captured video of a cardiac organoid into an image sequence;
setting an interrogation window size and a step size for a PIV algorithm to a first value for each of the converted image sequences;
configuring information on the cardiac organoid;
calculating a deformation velocity of the cardiac organoid using the first value, information on the cardiac organoid, and the PIV algorithm;
providing a real-time interactive tool for postprocessing of the deformation velocity;
calculating multiple parameters that assess contractility of the cardiac organoid on the basis of a profile of the postprocessed deformation velocity; and
outputting visual data and the multiple parameters used for analyzing contractility of the cardiac organoid, wherein
the first value is reset to a second value considering continuity and calculation time of a deformation velocity vector field calculated at the step of calculating the deformation velocity of the cardiac organoid.

2. The method according to claim 1, wherein as the second value, the PIV interrogation window size is set to 32 pixels, and the step size is set to 8 pixels.

3. The method according to claim 1, wherein an FFT algorithm is used to perform cross-correlation with interrogation windows in the PIV algorithm.

4. The method according to claim 1, wherein the postprocessing includes at least any one or more among setting a noise threshold, removing noise, smoothing, deleting noise peak points, and selecting or deselecting start, peak, and end points of contraction and relaxation.

5. The method according to claim 1, wherein the multiple parameters include at least any one or more among a maximum contraction force and a maximum relaxation force.

6. The method according to claim 4, wherein the multiple parameters further include at least any one or more among decay times of 90%, 50%, and 10% levels.

7. The method according to claim 1, wherein the visual data includes a contraction-relaxation velocity profile linked to an input image in real time, a beating image overlaid with the vector field of the deformation velocity, and a beating image of a heatmap form.

8. The method according to claim 1, wherein the image of the cardiac organoid is captured using a standard imaging device having a frame rate of 10 to 60 frames per second.

9. A PIV-based cardiac organoid contractility assessment system comprising:
a cardiac organoid image conversion unit for converting a captured image of a cardiac organoid into an image sequence;
a PIV setting unit for setting an interrogation window size and a step size for a PIV algorithm to a first value for each of the converted image sequences;
a cardiac organoid setting unit for setting information on the cardiac organoid;
a deformation velocity calculation unit for calculating a deformation velocity of the cardiac organoid using the first value, information on the cardiac organoid, and the PIV algorithm;
a postprocessing unit for providing a real-time interactive tool for postprocessing of the deformation velocity;
a multiple parameter calculation unit for calculating multiple parameters that assess contractility of the cardiac organoid on the basis of a profile of the postprocessed deformation velocity;
an output unit for outputting visual data and the multiple parameters used for analyzing contractility of the cardiac organoid; and
a control unit configured of software modules, a memory, and a processor needed for operation of a computer system, wherein
the first value is reset to a second value considering continuity and calculation time of a deformation velocity vector field calculated by the deformation velocity calculation unit for calculating the deformation velocity of the cardiac organoid.

10. The system according to claim 9, wherein as the second value, the PIV interrogation window size is set to 32 pixels, and the step size is set to 8 pixels.

11. The system according to claim 9, wherein an FFT algorithm is used to perform cross-correlation with interrogation windows in the PIV algorithm.

12. The system according to claim 9, wherein the postprocessing includes at least any one or more among setting a noise threshold, removing noise, smoothing, deleting noise peak points, and selecting or deselecting start, peak, and end points of contraction and relaxation.

13. The system according to claim 9, wherein the multiple parameters include at least any one or more among a maximum contraction force and a maximum relaxation force.

14. The system according to claim 13, wherein the multiple parameters further include at least any one or more among decay times of 90%, 50%, and 10% levels.

15. The system according to claim 9, wherein the visual data includes a contraction-relaxation velocity profile linked to an input image in real time, a beating image overlaid with the vector field of the deformation velocity, and a beating image of a heatmap form.

16. The system according to claim 9, wherein the image of the cardiac organoid is captured using a standard imaging device having a frame rate of 10 to 60 frames per second.

17. A computer program stored in a computer-readable recording medium, the computer program for executing the contractility assessment method according to claim 1.

18. A computer-readable recording medium storing computer programs, in which a computer program for executing the contractility assessment method according to claim 1 is recorded.
